(19) United States
(12) Patent Application Publication
(10) Pub. No.: US 2005/0004601 A1
(43) Pub. Date: Jan. 6, 2005

(54) Coded surgical aids

(76) Inventors: James Kam Fu Kong, Hong Kong (CN); Jimmy Wai Kwok Wong, Hong Kong (CN); Keith Dip Kei Luk, Hong Kong (CN)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/833,329

(22) Filed: Apr. 28, 2004

Related U.S. Application Data
(60) Provisional application No. 60/466,346, filed on Apr. 29, 2003.

(51) Int. Cl. A61L 17/00

(52) U.S. Cl. 606/228

(57) ABSTRACT

The present invention can provide a coded surgical suture, which is capable of affording a visual identification effect for determining the direction or end portions of the surgical suture. The surgical suture can have an elongated body member having first and second end portions. A coding member can be provided on one of the two end portions of the body member to afford a visual identification effect. In an exemplary embodiment, the coding member can have a color element. The present invention can also provide a coding system for identifying the direction or the end portions of a surgical suture or for distinguishing between different surgical sutures.

### CROSS-REFERENCE TO RELATED APPLICATION

This application claims the benefit of U.S. Provisional Patent Application No. 60/466,346 filed Apr. 29, 2003.

### FIELD OF THE INVENTION

The present invention generally relates to a surgical aid, such as a surgical suture. In particular, the present invention relates to a coded surgical suture. Moreover, the present invention relates to a system for coding various surgical aids, such as surgical sutures.

### BACKGROUND OF THE INVENTION

Surgical sutures are commonly used in various medical procedures, such as minimal invasive surgeries. Typically, one viewing portal and at least two working portals are needed when physicians use surgical sutures. With conventional surgical sutures, it is difficult to determine intraoperatively the movement direction of a suture due to the difficulty in identifying the correct suture end. Consequently, the wrong suture end can be pulled by mistake, which can cause the entire suture to be pulled out accidentally or undesirable twisting in the suture.

U.S. Pat. No. 3,642,003 discloses a suture having long-lasting germicidal properties. The sutures are rendered germicidal for long periods of time by contacting the suture with an aqueous solution of a cationic or anionic germicidal surface active agent such as a germicidal quaternary ammonium salt or a germicidal organic sulfonate to chemically bond the germicidal cation or anion of the salt to the proteinaceous substrate.

U.S. Pat. Nos. 3,674,901 and 3,807,273 each disclose a process for preparing the sutures. U.S. Pat. No. 3,807,273 discloses a method of making a pliable, dyed and braided polyester thread. The method comprises winding a polyester strand loosely around a perforated hollow dyeing element, passing dye under pressure through the winding of the polyester strand, hot-stretching the dyed strand to reduce the elastic memory thereof, coating the hot-stretched strand with a liquid, film-forming material adherent but substantially inert to said polyester strand, braiding a plurality of the coated filaments into a braided thread, and removing the film-forming material.

U.S. Pat. No. 3,987,797 discloses an antimicrobial surgical suture coated with an ionically bonded block elastomeric copolymer of polyquatemary polyurethane and a polyanionic polymer such as heparin. The coated suture is receptive to dyes for the preparation of colored sutures.

U.S. Pat. No. 4,470,941 discloses sutures comprising dissimilar synthetic polymer materials. U.S. Pat. No. 5,454,834 discloses a suture material having at least one inherently stable coil or loop along its length. U.S. Pat. No. 6,264,675 discloses a suture with an elongated body member having a needle and thread member disposed at one end and retention structure at the other end. U.S. Pat. No. 4,546,769 discloses a suture thread comprising a jacket or sheath of a tubular braided structure made of yams and a core located within the jacket containing crimped fibers. U.S. Pat. No. 5,766,188 discloses that a medical suturing material is loaded onto an automatic suturing device having a staple line when being used.

The present invention provides a coded surgical suture, which is capable of affording a visual identification effect for determining the direction or ends of the surgical suture. The present invention also provides a color coding system for identifying the direction or ends of a surgical suture.

### SUMMARY OF THE INVENTION

The present invention can provide a coded surgical suture, which is capable of affording a visual identification effect for determining the direction or ends of a surgical suture. The suture can comprise an elongated body member having first and second end portions. A coding member can be provided on one of the first and second end portions to distinguish one end portion from the other, affording a visual identification effect. In one embodiment, the coding member can comprise different first and second coding members formed on the end portions the body member to provide different visual effects.

The present invention can also provide a coding system for identifying the direction or the end portions of a surgical suture or for distinguishing between different surgical sutures.

### DETAILED DESCRIPTION OF THE INVENTION

For the purposes of promoting an understanding of the principles of the present invention, various exemplary embodiments will now be discussed in great details.

The present invention can provide a coded surgical aid, such as a surgical suture. In one embodiment, the surgical aid can comprise a body member extending between a first end portion and a second end portion. A coding member can be provided to be affixed to at least one of the first and second end portions and extending toward the other of the end portions to distinguish the first end portion from the second end portion. For example, the coding member is capable of making the first and second end portions to exhibit different visual effects. The present invention can also provide a coding system for identifying the direction or end portions of a surgical suture or for distinguishing between different surgical sutures.

In one exemplary embodiment, the surgical suture can comprise a body member having first and second end portions. The body member can have various shapes, lengths, diameters, and/or cross-sections. For example, the body member can be an elongated body member having a length between the two end portions. In one exemplary embodiment, the body member can be a thread member. In an exemplary embodiment, the thread member can be formed of a single strand. In another exemplary embodiment, the body member can be formed of multi-filament strands. For example, the multi-filament strands can be twisted or braided to form the body member.

Additionally or alternatively, the body member can be made of various materials. For example, such as natural materials (e.g., silk) or synthetic materials (e.g., polyester). In one exemplary embodiment, the body member can be made of a flexible material. It will be appreciated that other embodiments of the body member are also within the scope of the present invention.

In one embodiment, the surgical suture can comprise a coding member provided on one of the end portions of the body member. For example, the coding member can be affixed to a first end portion of the surgical suture. In one exemplary embodiment, the coding member can extend for about 1% to about 99% of the entire length of the body member. In an exemplary embodiment, the coding member can extend from one end portion to the middle portion of the body member. In another exemplary embodiment, different first and second coding members can be provided at both end portions of the body member to provide a differential visual identification effect. In an exemplary embodiment, the first and second coding members can extend from the respective first and second end portions to the middle portion of the body member.

The coding member can be in various forms, to provide a visual differentiation and/or identification effect. In one exemplary embodiment, the coding member can comprise various color elements, such as to afford an apparent visual contrasting effect. For example, the coding member can comprise a color element of red, green, orange, blue, and etc. In an exemplary embodiment, the coding member can be a single color element. In another exemplary embodiment, the color element can be formed in various ways, such as colored at a different and/or variable extent. For example, the color element can have a gradation of colors, multiple colors, a varying intensity, or a design pattern. In one exemplary embodiment, the color element can comprise a gradual change in color intensity, such as to provide a gradation of color change. For example, the color intensity can decrease from the end portion toward the middle portion of the body member. The color changes can assist the surgeon to visually identify the direction of the suture and/or the location of an end portion during a medical procedure, such as an arthroscopic or endoscopic operation. It will be appreciated that other embodiments of the color element are also within the scope of the present invention.

In another exemplary embodiment, the coding member can be formed to comprise a plurality of coding sections. For example, the plural coding sections can have different color elements, lengths, and the like. In an exemplary embodiment, first and second coding sections can be formed to have different colors from each other. In another exemplary embodiment, a plurality of coding sections can be provided to be positioned at various intervals along the length of the elongated body member, such as to achieve an apparent visual contrasting effect. In one exemplary embodiment, the plural coding sections can be positioned at one end portion of the elongated body member. It will be appreciated that other embodiments of the coding sections are also within the scope of the present invention.

In a further exemplary embodiment, the coding member can be formed to have various coding patterns, such as of shapes, lengths, or arrangement, to afford a different identification effect. In an exemplary embodiment, the coding member can have a spiral pattern extending along the body member. In another exemplary embodiment, the coding member can comprising a plurality of coding sections where the spacing between the adjacent coding sections can increase from one end portion toward the other end portion of the body member. In a further exemplary embodiment, the plural coding sections can have different colors or gradation of colors. It will be appreciated that other embodiments of the coding patterns are also within the scope of the present invention.

The coding member can be formed by various conventional methods. For example, the coding member can be coated, sprayed, glued, dyed, stained, or otherwise affixed onto the body member. In an exemplary embodiment, the color element can be formed by a dying process. For example, gradation of change in color intensity can be employed in a dying process to form a color element. In another exemplary embodiment, the coding member can be integrally formed with the body member. It will be appreciated that other embodiments of forming the coding member are also within the scope of the present invention.

Although the above description is made in connection with an exemplary surgical suture, it will be appreciate that the present invention is applicable to various other surgical aids.

According to another aspect of the present invention, a coding system can be provided to distinguish between a plurality of surgical aids. For example, the coding system can comprise a plurality of surgical aids each having a body member extending between two end portions, such as described above. In an exemplary embodiment, the first end portion of each of the surgical aids can be identified with a first coding member. For example, the surgical aids are color coded. In one exemplary embodiment, the first coding members can differ from one another. Additionally or alternatively, the second end portion of each of the surgical aids can be identified with a second coding member. In an exemplary embodiment, the second coding members can differ from one another. It will be appreciated that other embodiments of the coding system are also within the scope of the present invention.

In anther exemplary embodiment, the different first coding members can be predetermined or otherwise recorded for users' reference. For example, the coding system can enable a user to identify each surgical aid even when more than one surgical aides are used. Additionally or alternatively, the coding system can assist a user to match a first surgical aid with a second surgical aid by following the coding scheme.

Optionally, the surgical aids can comprise various accessory elements to facilitate to manipulate the surgical aids. For example, an anchor or a needle can be provided to one or both end portions of a surgical suture to facilitate the formation of a knot. It will be appreciated that other embodiments of the surgical suture are also within the scope of the present invention.

It will be appreciated that the various features described herein may be used singly or in any combination thereof. Therefore, the present invention is not limited to only the embodiments specifically described herein. While the foregoing description and drawings represent a preferred embodiment of the present invention, it will be understood that various additions, modifications, and substitutions may be made therein without departing from the spirit and scope of the present invention as defined in the accompanying claims. In particular, it will be clear to those skilled in the art that the present invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. The presently disclosed embodiment is therefore to be considered in all respects as illustrative and not restrictive, the scope of the invention being indicated by the appended claims, and not limited to the foregoing description.

1. A surgical suture comprising:
a body member extending between a first end portion and a second end portion; and
a coding member affixed to at least one of the first and second end portions and extending toward the other of the end portions;
wherein the coding member distinguishes the first end portion from the second end portion.

2. The surgical suture of claim 1, wherein the first and second end portions exhibit different visual effects.

3. The surgical suture of claim 1, wherein the coding member is integrally formed with the body member.

4. The surgical suture of claim 1 being made of a flexible material.

5. The surgical suture of claim 1, wherein the coding member extends for about 1% to about 99% of the length of the body member.

6. The surgical suture of claim 11, wherein the coding member extends to a middle portion of the body member.

7. The surgical suture of claim 1, wherein
the first and second end portions are provided with first and second coding members, respectively; and
the first and second coding members differ from each other.

8. The surgical suture of claim 7, wherein the first and second coding members extend from the first and second end portions, respectively, to a middle portion of the body member.

9. The surgical suture of claim 1, wherein the coding member comprises a color element.

10. The surgical suture of claim 9, wherein the coding member comprises one single color element.

11. The surgical suture of claim 10, wherein the color element has a varying color intensity.

12. The surgical suture of claim 10, wherein the color element has a color gradation.

13. The surgical suture of claim 1, wherein the coding member comprises a plurality of coding sections.

14. The surgical suture of claim 13, wherein the coding member comprises a plurality of color elements.

15. The surgical suture of claim 1 further comprising an accessory element to facilitate the formation of a knot.

16. A coded surgical aid comprising:
a body member extending between two end portions; and
a coding member affixed to a first end portion and extending toward a second end portion;
wherein the coding member distinguishes the first end portion from the second end portion.

17. The coded surgical aid of claim 16 being a surgical suture.

18. A coding system for surgical aids, comprising
providing a plurality of surgical aids each having a body member extending between two end portions; and
identifying a first end portion of each surgical aid with a first coding member;
wherein the plurality of surgical aids have different first coding members.

19. The coding system of claim 18 comprising identifying a second end portion of each surgical aid with a second coding member.

20. The coding system of claim 18, wherein the surgical aids are color coded.
